SEQUENCE LISTING (15 sequences)

SEQ ID NO: 1 - DNA, 20 bp, Artificial
  Aggrecanase 1 forward primer
actggtggtggcagatgaca

SEQ ID NO: 2 - DNA, 23 bp, Artificial
  Aggrecanase 1 reverse primer
tcactgttagcaggtagcgcttt

SEQ ID NO: 3 - DNA, 19 bp, Artificial
  Aggrecanase 1 probe
tggccgcattccacggtgc

SEQ ID NO: 4 - DNA, 19 bp, Artificial
  Aggrecanase 2 forward primer
tggctcacgaaatcggaca

SEQ ID NO: 5 - DNA, 23 bp, Artificial
  Aggrecanase 2 reverse primer
ggaaccaaaggtctcttcacaga

SEQ ID NO: 6 - DNA, 25 bp, Artificial
  Aggrecanase 2 probe
cttggcctctcccatgacgattcca

SEQ ID NO: 7 - DNA, 21 bp, Artificial
  Collagenase 1 forward primer
gggagatcatcgggacaactc

SEQ ID NO: 8 - DNA, 19 bp, Artificial
  Collagenase 1 reverse primer
gggcctggttgaaaagcat

SEQ ID NO: 9 - DNA, 29 bp, Artificial
  Collagenase 1 probe
ccttttgatggacctggaggaaatcttgc

SEQ ID NO: 10 - DNA, 20 bp, Artificial
  Collagenase 3 forward primer
tggcattgctgacatcatga

SEQ ID NO: 11 - DNA, 17 bp, Artificial
  Collagenase 3 reverse primer
gccagagggcccatcaa

SEQ ID NO: 12 - DNA, 30 bp, Artificial
  Collagenase 3 probe
ctcttttggaattaaggagcatggcgactt

SEQ ID NO: 13 - DNA, 21 bp, Artificial
  Stromelysin forward primer
ttctcgttgctgctcatgaaa

SEQ ID NO: 14 - DNA, 26 bp, Artificial
  Stromelysin reverse primer
tagagtgggtacatcaaagcttcagt

SEQ ID NO: 15 - DNA, 25 bp, Artificial
  Stromelysin probe
tggccactccctgggtctctttcac

(19) United States
(12) Patent Application Publication
(10) Pub. No.: US 2005/0074744 A1
(43) Pub. Date: Apr. 7, 2005

(54) Assays for evaluating anti-osteoarthritic activity

(76) Inventor: Peter DiBenedetto, New Bedford, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 10/675,971

(22) Filed: Oct. 2, 2003

(51) Int. Cl. C12Q 1/00; C12Q 1/68; C12N 5/08

(52) U.S. Cl. 435/4; 435/366; 435/6

(57) ABSTRACT

A novel assay for evaluating the efficacy of test compounds in preventing or treating osteoarthritic cartilage damage is provided. Using an in vitro articular cartilage graft system, a test compound is administered and its effect on the expression of a number of osteoarthritic marker molecules is measured. Changes in the levels of these molecules after administration of the test compound indicates an effect on osteoarthritic cartilage damage.

### FIELD OF THE INVENTION

This invention relates to in vitro assays for identification or characterization of therapeutic agents useful for prevention or treatment of joint diseases, such as osteoarthritis. The invention further relates to compositions for use in the in vitro assays and methods of making those compositions.

### BACKGROUND OF THE INVENTION

Articular cartilage, also called hyaline cartilage, is an avascular tissue found-at the end of-articulating bones. Articular cartilage is composed primarily of collagens and proteoglycans. The major collagens in hyaline cartilage are type II, IX, and XI collagens. The major proteoglycan found in hyaline cartilage is aggrecan. Smaller amounts of biglycan and decorin are also present in hyaline cartilage.

Osteoarthritis is the breakdown and eventual loss of articular cartilage. Damage to articular cartilage by excessive wear, traumatic injury, or disease eventually leads to osteoarthritis. The condition is characterized by a loss of cartilage matrix proteins, such as collagens and proteoglycans. The loss of these matrix proteins is induced by the upregulation of metalloproteinases, for example, collagenase and aggrecanase. Osteoarthritis is also characterized by increased expression of inflammatory molecules, such as interleukins and TNF family members.

The are no methods currently available for repairing osteoarthritis-induced cartilage damage. Attempts to repair cartilage tissue induced by arthritis, i.e., microfracture or abrasion, result in the formation of fibrocartilage in place of the damaged hyaline articular cartilage. Fibrocartilage is not a suitable replacement for natural hyaline cartilage because the major collagen in fibrocartilage, type I collagen, has a weaker structure than type II collagen, the major collagen in hyaline cartilage. Thus, fibrocartilage is inferior to the original hyaline cartilage in its long-term ability to cushion joints and maintain smooth articulating surfaces. Because the repair of articular hyaline cartilage is so difficult, the prevention of the progressive degeneration of articular cartilage is an important goal in the effective treatment of cartilage injury and osteoarthritis.

Several in vitro cartilage tissue culture systems have been developed to evaluate therapeutic compositions that may-prevent-or-slow hyaline cartilage degeneration in a patient. These include chondrocyte monolayer cultures (Tubo & Brown, Human Cell Culture 5:1-16 (2001)), three-dimensional alginate cultures (Guo et al., Connective Tissue Research 19:277-97 (1988)), pellet cultures (e.g., monolayer cultures with very high cell densities) (Kato et al., Proc Nat Acad Sci USA 85:9552-9556 (1988)), and cartilage explants (Muir et al., Drugs 35(S1):15-23 (1988)). Although an osteoarthritic phenotype can be induced in chondrocyte monolayer cultures or explant systems by treatment with inflammatory cytokines such as IL-1α, IL-1β, and TNFα, these systems have certain disadvantages. First, monolayer cultures are de-differentiated and lack the necessary three-dimensional structure of native cartilage. They express type I collagen instead of type II collagen, and therefore, do not present a gene expression profile mimicking that of native articular cartilage. Alginate and pellet cultures, while having a three-dimensional structure, do not form a tissue resembling native cartilage. Human explant cultures require human tissue donors, which are often in short supply. Explants also contain limited amounts of RNA, making quantitative measurement of RNA levels difficult.

Articular cartilage grafts (ACGs), such as those described in U.S. Pat. No. 5,326,357, have been developed for implantation into patients to repair articular cartilage damage. ACGs are generally synthesized by isolating chondrocytes from bovine or porcine tissue and growing them on filters. However, it is also possible to grow ACGs from human chondrocytes. Prior to this invention, and despite success in initial preclinical applications, the ACG system had not been used as an in vitro model of osteoarthritis.

Due to the insufficiencies of the in vitro tissue and cell culture systems currently used to model osteoarthritic cartilage damage, there is still a need to provide in vitro assay systems that closely mimic an osteoarthritic disease state and are amenable to measurement of disease parameters.

### SUMMARY OF THE INVENTION

The present invention provides an in vitro model of osteoarthritic cartilage damage. The invention further provides an assay for evaluating the efficacy of a test compound in treating or preventing osteoarthritic cartilage damage.

One aspect of the invention is a method for making an ACG having cartilage damage characteristic of osteoarthritis (e.g., expressing an osteoarthritic phenotype). The method comprises inducing osteoarthritic cartilage damage in the ACG. In some embodiments, the method comprises administering an effective amount of one or more cytokines to an ACG to induce osteoarthritic cartilage damage in the ACG. In certain embodiments, the cytokine is IL-1β, IL-1α, and/or TNFα.

Another aspect of the invention is a composition comprising an ACG having osteoarthritic cartilage damage. In certain embodiments, the osteoarthritic cartilage damage is induced in the ACG by treating it with an effective amount of a composition comprising one or more cytokines, such as, for example, IL-1β, IL-1α, or TNFα.

Another aspect of the invention is an assay for evaluating the efficacy of a test compound for treating or preventing osteoarthritic cartilage damage. The method comprises: (1) administering an osteoarthritic phenotype inducing composition to an ACG; (2) administering a test compound to the ACG; and (3) comparing the change in the levels of one or more osteoarthritic markers in an ACG before and after administration of an osteoarthritic phenotype inducing composition alone with the change in the levels of the same marker(s) in an ACG before and after administration of both the test compound and the osteoarthritic phenotype inducing composition; wherein a lower level of change in the ACG to which the test compound was administered indicates that the test compound is efficacious for treating or preventing osteoarthritic cartilage damage.

In certain embodiments, an effective amount of one or more cytokines such as, for example, IL-1β, IL-1α, and TNFα, is administered as the osteoarthritic cartilage damage inducing compound.

Additional features and advantages of the invention will be set forth in part in the description which follows, and in part will be obvious from the description, or may be learned by practice of the invention.

### BRIEF DESCRIPTION OF THE DRAWINGS

FIGS. 1A-1D show a toluidine blue stain of ACGs with and without incubation with IL-1β and/or debromohymenialdesine (DBH). All samples were harvested at 24 hours. FIG. 1A depicts an untreated ACG. FIG. 1B depicts an ACG incubated with IL-1β alone. FIG. 1C depicts an ACG incubated with both IL-1β and DBH. FIG. 1D depicts an ACG incubated with IL-1β and DMSO.

FIG. 2 shows levels of collagenase 1 RNA in ACGs before and after incubation with IL-1β and/or DBH. The level of collagenase 1 RNA in Sample 1 was measured at the onset of the experiment. RNA levels in the other samples were measured after 24 hours. Sample 2 was untreated ACG. Sample 3 was incubated with IL-1β alone. Sample 4 was incubated with IL-1β and DBH. Sample 5 was incubated with IL-1β and DMSO.

FIG. 3 shows levels of collagenase 3 RNA in ACGs before and after incubation with IL-1β and/or DBH. The level of collagenase 3 RNA in Sample 1 was measured at the onset of the experiment. RNA levels in the other samples were measured after 24 hours. Sample 2 was untreated ACG. Sample 3 was incubated with IL-1β alone. Sample 4 was incubated with IL-1β and DBH. Sample 5 was incubated with IL-1β and DMSO.

FIG. 4 shows levels of stromelysin RNA in ACGs before and after incubation with IL-1β and/or DBH. The level of stromelysin RNA in Sample 1 was measured at the onset of the experiment. RNA levels in the other samples were measured after 24 hours. Sample 2 was untreated ACG. Sample 3 was incubated with IL-1β alone. Sample 4 was incubated with IL-1β and DBH. Sample 5 was incubated with IL-1β and DMSO.

FIG. 5 shows levels of aggrecanase 1 RNA in ACGs before and after incubation with IL-1β and/or DBH. The level of aggrecanase 1 RNA in Sample 1 was measured at the onset of the experiment. RNA levels in the other samples were measured after 24 hours. Sample 2 was untreated ACG. Sample 3 was incubated with IL-1β alone. Sample 4 was incubated with IL-1β and DBH. Sample 5 was incubated with IL-1β and DMSO.

FIG. 6 shows levels of aggrecanase 2 RNA in ACGs before and after incubation with IL-1β and/or DBH. The level of aggrecanase 2 RNA in Sample 1 was measured at the onset of the experiment. RNA levels in the other samples were measured after 24 hours. Sample 2 was untreated ACG. Sample 3 was incubated with IL-1β alone. Sample 4 was incubated with IL-1β and DBH. Sample 5 was incubated with IL-1β and DMSO.

### DETAILED DESCRIPTION OF THE EMBODIMENTS

In order for the present invention to be more readily understood, certain terms are defined herein. Additional definitions are set forth throughout the detailed description.

The terms “osteoarthritic phenotype” and “osteoarthritis” refer to a degeneration of cartilage, chondrocytes, and/or bone, either in vivo or in vitro. The term “osteoarthritic cartilage damage” refers to a degeneration of cartilage and/or chondrocytes in vivo or in vitro. This degeneration can be induced by, for example, cytokines, and is characterized by upregulation of proteinase expression and the resulting loss of proteoglycans (Kolibas et al., “Effect of cytokines and anti-arthritic drugs on glycosaminoglycan synthesis by bovine articular chondrocytes,” Agents and Actions, 27(3/4):245-249 (1989)).

The term “osteoarthritic marker” refers to any molecule that has an altered level of expression or function in an ACG expressing an osteoarthritic phenotype as compared to the level of expression or function in an ACG made of normal cartilage cells or tissue. The altered expression or function may be a cause or a result of the cartilage damage.

The term “articular cartilage” refers to any cartilage naturally present at articulating surfaces in vivo, or any cartilaginous tissue derived in vivo or in vitro from the precursor chondrocytes of native articular cartilage or from undifferentiated stem cells.

The term “articular cartilage graft” or “ACG” refers to a composition produced by the in vitro system of inducing the growth of articular cartilage tissue, as described in, e.g., U.S. Pat. No. 5,326,357, from precursor chondrocytes or alternatively, from undifferentiated stem cells.

The term “test compound” refers to any compound or composition, chemical or biological, to be evaluated in the methods of the invention.

The terms “therapeutic compound” and “therapeutic,” as used herein, refer to any compound capable of treating, reversing, ameliorating, halting, slowing progression of, or preventing clinical manifestations of a disorder, or of producing a desired biological outcome.

The term “anti-osteoarthritic activity” refers to any activity that prevents or reduces the manifestations of the osteoarthritic phenotype.

The invention is based, in part, on the recognition that ACGs have certain advantages over presently used in vitro tissue culture systems such as, e.g., monolayer, alginate, and explant systems. The structure of the tissue formed with ACGs resembles native cartilage. Chondrocytes growing within the ACG construct are in a differentiated state, thus allowing for the expression of proteins that are characteristic of articular cartilage, like type II collagen and aggrecan. The invention is also based, in part, on the discovery that administration of IL-1β to articular cartilage grafts (ACGs) induces osteoarthritic-like cartilage damage. More specifically, administration of IL-1β to ACGs causes the loss of proteoglycans and collagen, as determined by histological staining. The invention is further based, in part, on the discovery that administration of debromohymenialdesine (DBH), an anti-osteoarthritic agent, to ACGs reduces the loss of proteoglycans and collagen and prevents the IL-1β-induced degeneration of the ACG.

Certain embodiments of the invention are based, in part, on the discovery that administration of IL-1β to ACGs increases the levels of RNAs encoding osteoarthritis-linked inflammatory molecules. Additional embodiments are based, in part, on the discovery that administration of IL-1β to ACGs increases the levels of RNAs encoding osteoarthritis-linked proteinases.

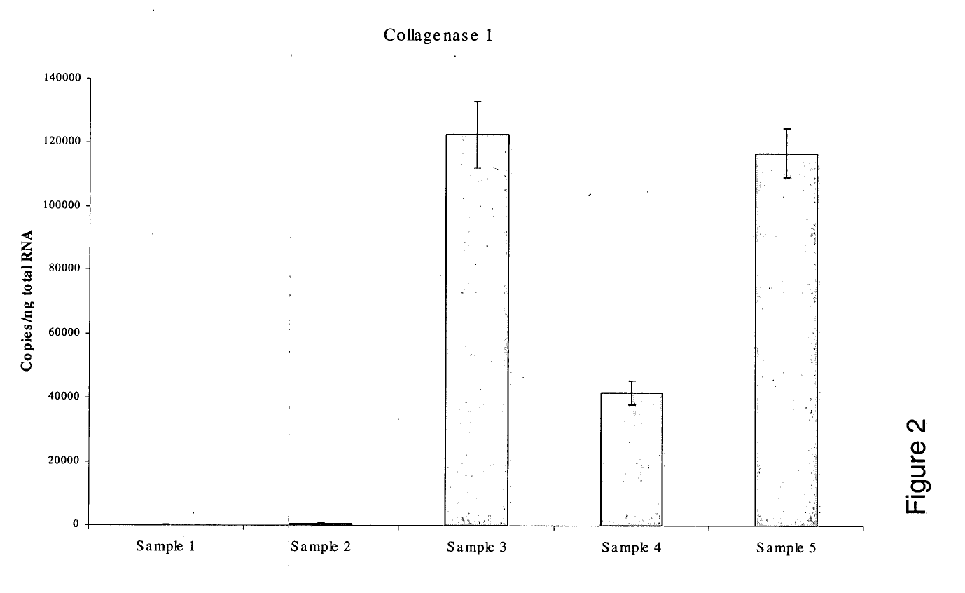

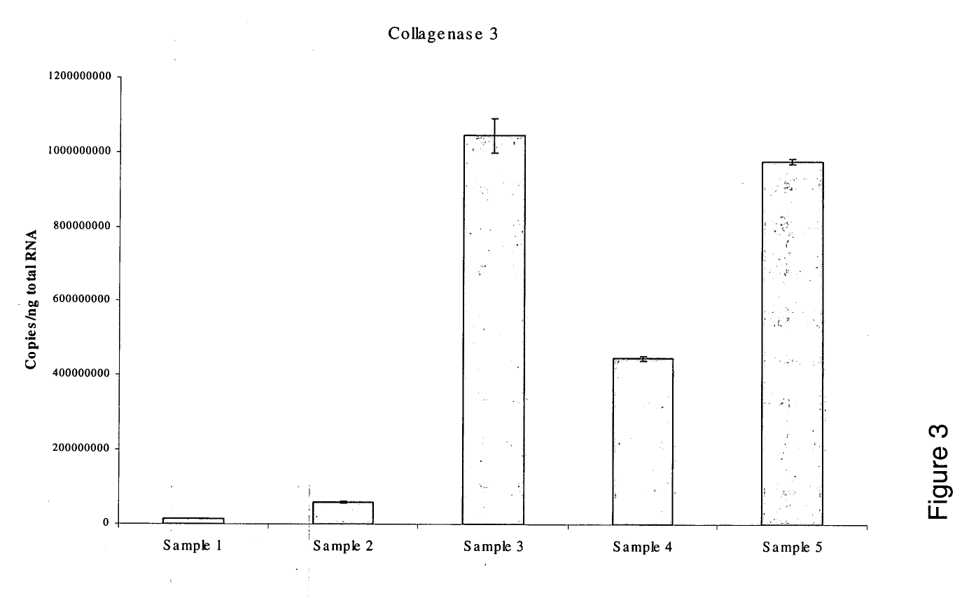

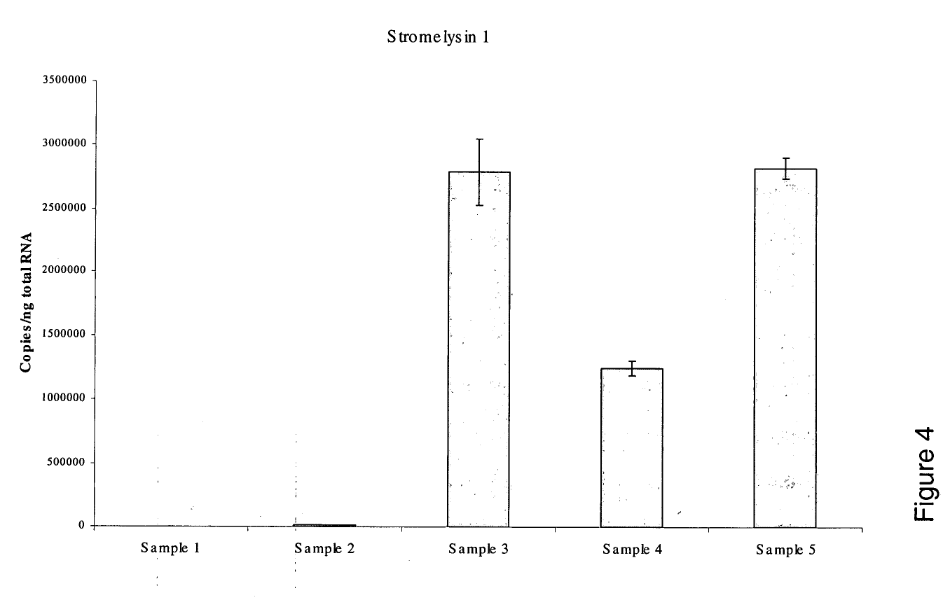

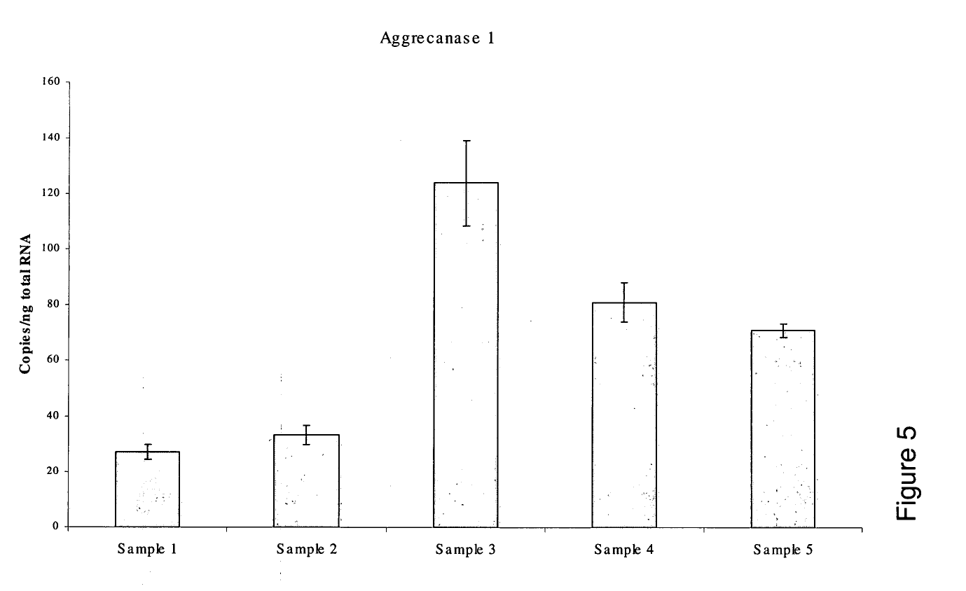

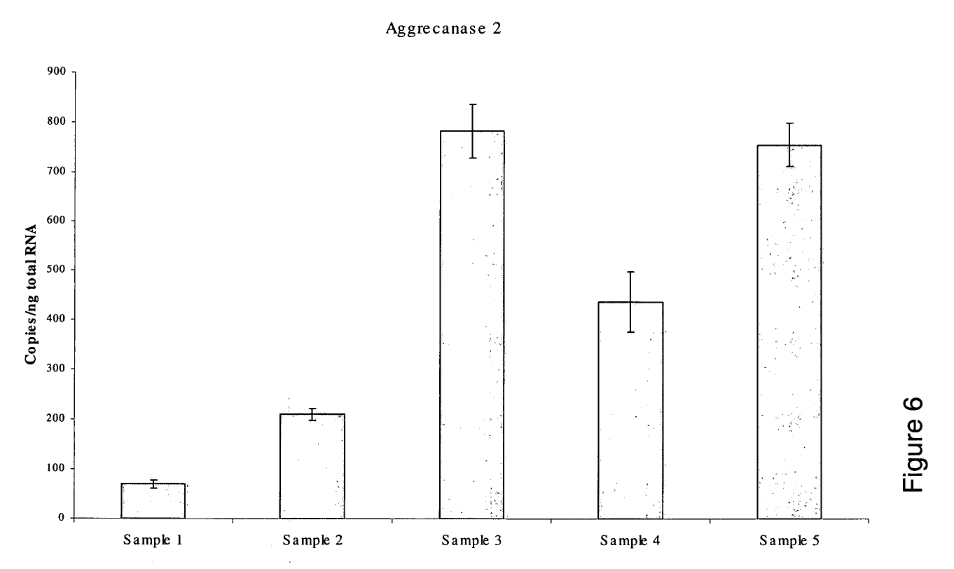

Additionally, certain embodiments are based, in part, on the discovery that administration of DBH to the IL-1β-treated ACGs reduces the IL-1β-induced upregulation of certain RNAs encoding osteoarthritis-linked proteinases (see, e.g., FIGS. 2-6). DBH and its anti-osteoarthritic activity are described in U.S. Pat. No. 5,591,740. DBH has anti-osteoarthritic activity in both in vitro and in vivo models, including chondrocyte monolayer cultures and the Hartley guinea pig model of osteoarthritis. Specifically, DBH inhibits IL-1 induced degradation of matrix proteins in monolayer cultures and reduces preexisting cartilage damage in the guinea pig model. Therefore, DBH is a suitable test compound to establish the efficacy of certain embodiments of the invention.

ACG Expressing an Osteoarthritic Phenotype

Methods for making an ACG with cartilage damage characteristic of osteoarthritis (i.e., expressing an osteoarthritic phenotype) comprise inducing osteoarthritic cartilage damage in the ACG by administering an amount of one or more cytokines effective to induce osteoarthritic cartilage damage. In certain embodiments, the cytokine administered to the ACG is IL-1β, IL-1α, and/or TNFα. The cytokines used in the methods of the invention can be administered at concentrations ranging from 1 ng/ml to 1 mg/ml. For example, they can be administered at concentrations ranging from 0.1 μg/ml to 100 μg/ml, 0.5 μg/ml to 10 μg/ml, or 1 μg/ml to 5 μg/ml. Other cytokines may be used as well and are known to those skilled in the art. Using the methods described herein, the ability of any known cytokine to induce osteoarthritic cartilage damage can be readily determined.

The invention also provides compositions comprising an ACG expressing an osteoarthritic phenotype. In particular embodiments, the osteoarthritic cartilage damage has been induced in the ACG by administering one or more cytokines, such as, for example, IL-1β, IL-1α, or TNFα. In other embodiments, the osteoarthritic cartilage damage is present in the ACG without administration of cytokines. Other embodiments of the invention include lysates, conditioned media, and chondrocytes produced or isolated from ACGs expressing an osteoarthritic phenotype.

Assay for Anti-Osteoarthritic Compounds

Another aspect of the invention provides an assay for evaluating the efficacy of a test compound for treating or preventing osteoarthritic cartilage damage. The assay comprises: (1) administering an osteoarthritic phenotype inducing composition to an ACG; (2) administering a test compound to the ACG; and (3) comparing the change in the levels of one or more osteoarthritic markers in an ACG before and after administration of an osteoarthritic phenotype inducing composition alone with the change in the levels of the same markers in an ACG before and after administration of both the test compound and the osteoarthritic phenotype inducing composition; wherein a. lower level of change in the ACG to which the test compound was administered indicates that the test compound is efficacious for treating or preventing osteoarthritic cartilage damage.

In certain embodiments, the assay comprises (1) culturing three ACGs; (2) measuring the levels of selected osteoarthritic markers in the first ACG prior to any treatment in order to establish a baseline level of the selected marker(s) in normal cartilage cells or tissue; (3) administering an osteoarthritic phenotype inducing composition to the second ACG and measuring the level of osteoarthritic marker(s) after the onset of the osteoarthritic phenotype; (4) administering both an osteoarthritic phenotype inducing composition and a test compound to the third ACG and measuring the level of an osteoarthritic marker(s) in the ACG; and (5) comparing the change in the levels of the selected osteoarthritic marker(s) in the three ACGs. If the magnitude of the change between the first two ACGs (untreated and treated only with an osteoarthritic phenotype inducing composition) is greater than the change between the first and third ACGs (untreated and treated with both an osteoarthritic phenotype inducing composition and a test compound) then the test compound is efficacious for treating or preventing osteoarthritic cartilage damage.

It will apprciated by those of skill in the art that the level of the osteoarthritic marker(s) in normal ACGs and ACGs treated only with an osteoarthritic inducing composition can be determined at the same time as the level of the marker(s) in ACGs incubated with an osteoarthritic phenotype inducing composition and a test compound, or they can be predetermined. In either case, the difference in the change of the level of the marker(s) from normal ACG to the level of the marker(s) after administration of an osteoarthritic phenotype inducing composition and a test compound is compared to the difference in the level of the marker(s) from normal ACG to the level of the marker(s) in ACG after administration of the osteoarthritic inducing composition alone.

It will also be appreciated by those of skill in the art that the level of certain osteoarthritic markers will increase in the presence of osteoarthritic cartilage damage, while the levels of other markers will decrease. For example, matrix proteins are degraded during osteoarthritic cartilage damage, reducing the protein levels of these markers. As a result, in some embodiments of the invention, a test compound will be considered efficacious if the levels of one or more osteoarthritic markers in an ACG incubated with both the test compound and an osteoarthritic phenotype inducing composition are above the levels of the same marker in an ACG incubated with the osteoarthritic phenotype inducing composition alone.

In contrast, both the RNA and protein levels of metalloproteinases and inflammatory molecules increase in an ACG expressing an osteoarthritic phenotype. As a result, in some embodiments of the invention, a test compound will be considered efficacious if the levels of one or more osteoarthritic markers in an ACG incubated with both the test compound and an osteoarthritic phenotype inducing composition are below the levels of the same marker in an ACG incubated with the osteoarthritic phenotype inducing composition alone.

The magnitude of change between the expression levels of the osteoarthritic marker in ACGs expressing the osteoarthritic phenotype and those not expressing the phenotype can be at least 5%, 10%, 25%, 50%, 75%, or 100%. The magnitude of change considered to be significant for indicating the efficacy of a test compound will depend on both the method used to induce the osteoarthritic phenotype and the test compound itself. As previously noted, the level of osteoarthritic marker(s) in untreated ACGs and ACGs incubated with osteoarthritic phenotype inducing compositions alone can be predetermined or, alternatively, can be determined concurrently with the level of osteoarthritic marker(s) in ACGs incubated with the test compound.

Suitable osteoarthritic markers include matrix proteins. In some embodiments, the matrix proteins are collagens or proteoglycans. In particular embodiments, the collagen is type II collagen and the proteoglycan is aggrecan. Matrix protein markers for osteoarthritic cartilage damage can be measured in the assays of the invention by, e.g., histological evaluation.

Other suitable osteoarthritic markers include metalloproteinases. One or more metalloproteinases selected from the group consisting of collagenase 1, collagenase 3, stromelysin 1, aggrecanase 1, and aggrecanase 2 may be used in the assays of the invention. Inflammatory molecules, such as, for example, TNF, IL-6, IL-8, IL-1β, nitric oxide synthase 2A, prostaglandin-endoperoxidase synthase 2, and NF-κB, may also be used as osteoarthritic markers in the assays of the invention.

Additional osteoarthritic markers suitable for use in the assays of the invention may be identified with known techniques, such as microarray analysis or differential expression cloning. DNA Microarrays: A Molecular Cloning Manual (Bowtell & Sambrook) Cold Spring Harbor Laboratory Press, 2002; and Liang, P. et al. “Method of differential display” Methods in Mol. Genetics, 5:3-16 (1994).

In certain embodiments of the assays of the invention, the test compound and the osteoarthritic phenotype induction composition are administered at the same time. In other embodiments, the test compound is administered first. In another embodiment the test compound is administered second. The amount of time between the administration of the test compound and the osteoarthritic phenotype inducing composition will vary with the stability of the test compound and the osteoarthritic cartilage damage inducing compound (e.g., about at least 1 minute, 15 minutes, 30 minutes, 1 hour, 3 hours, 6 hours, 12 hours, 24 hours, 36 hours, or 72 hours, or about at most 72 hours, 36 hours, 24 hours, 12 hours, 6 hours, or 2 hours).

The determination of the appropriate amounts of cytokine and test compound to administer can be determined by routine experimentation. Typical methods for determining the appropriate amount of cytokines or test compounds include determining the levels of the composition naturally present in native tissue, review of the literature for similar experiments, or dose response curve experiments.

The test compound can be preselected or be part of a larger scale screening of compounds. The methods and assays of the invention can be used to screen panels of test compounds or to confirm the anti-osteoarthritic activity of a known potential anti-osteoarthritic therapeutic composition. The test compound may be part of a library of compounds of interest, or it may be part of a library of structurally-related compounds. The structure of the compound may be known or unknown. Test compounds may be predetermined by known functions or structures. For example, a test compound may be chosen because it binds to a cytokine or has a known anti-metalloproteinase activity. Additionally, a test compound may be selected because of its homology to a known anti-osteoarthritic, anti-cytokine, or anti-proteinase compound. Alternatively, selection of the test compound can be arbitrary. In non-limiting examples, the test compound may be a peptide, a protein or protein fragment, a small organic molecule, a chemical composition, a nucleic acid, or an antibody. A number of methods for evaluating the appropriateness of a test compound are well known.

In certain embodiments of the invention, the levels of osteoarthritic markers are measured at the RNA level. In some embodiments, the RNA levels of the marker are determined by quantitative real time PCR (Q RT-PCR). In other embodiments, the RNA levels of the marker are determined by Northern blot or another method for determining RNA levels. For example, methods for designing probes and quantitating RNA levels by Northern blot are described in, e.g., Cloning: A Laboratory Manual, 2^{nd} edition. (Sambrook, Fritch, and Maniatis, eds.), Cold Spring Harbor Laboratory Press, 1989.

In other embodiments, the levels of osteoarthritic markers are measured at the protein level. In some embodiments, the protein levels can be determined by histological analysis, Western blot, ELISA, immunohistochemistry, or any other method for determining protein levels. The assays will be performed, for example, according to Current Protocols in Molecular Biology (Ausubel et. al., eds.) New York: John Wiley and Sons, 1998. However, as these assays are well known, it will be apparent that they can be performed a number of different ways while still obtaining the same result of determination of protein levels. Antibodies are suitable reagents for most of the protein level assays. Appropriate antibodies are readily obtainable. For example, an antibody to stromelysin 1 can be purchased from Triple Point Biologics (Cat. No. RP5MMP 3). This polyclonal antibody can be used for the above-mentioned assays when accompanied by suitable secondary antibodies, which would be easily identifiable and obtainable by those skilled in the art. Production of the appropriate antibody is also possible by expressing the protein of interest in vitro and raising antibodies to the protein or protein fragment in an appropriate organism, such as, for example, a rabbit, mouse, or goat.

The levels of osteoarthritic markers can be measured in combination or alone. One advantage of the ACG system over other cartilage model systems is that the proteins and RNAs expressed in ACGs are the same as those expressed in native articular cartilage. Therefore, for example, it is possible to measure the protein levels of a matrix protein and the RNA levels of a metalloproteinase in the same assay. Accordingly, another aspect of the invention is the determination of the levels of combinations of osteoarthritic markers. In some embodiments, the protein levels of a matrix protein and the RNA levels of a metalloproteinase or an inflammatory molecule are determined. In certain embodiments, the protein levels of type II collagen and/or aggrecan and the RNA levels of collagenase 1, collagenase 3, stromelysin 1, aggrecanase 1, aggrecanase 2, TNF, IL-6, IL-8, IL-1β, nitric oxide synthase 2A, prostaglandin-endoperoxidase synthase 2, and/or NF-κB are determined. In other embodiments, both the protein levels and the RNA levels of metalloproteinases and/or inflammatory molecules are determined. In yet other embodiments, the protein levels of a matrix protein and the protein levels of a metalloproteinase and/or inflammatory molecule are determined.

It will be appreciated by those of skill in the art that there are many ways to carry out the assay of the invention to evaluate the efficacy of a test compound in treating osteoarthritis. Typically, for example, an ACG of human or animal origin is grown from precursor chondrocytes, which can be freshly isolated from cartilage tissue or expanded by cell culture in vitro. Precursor chondrocytes may also be isolated from bone marrow. This ACG should be cultured for approximately three weeks or until it contains ratios of matrix proteins (particularly the aggrecan and collagen type II to collagen type I ratios) that are similar to the ratios found in native hyaline cartilage. The culture medium will have the appropriate salts and buffers to allow growth of the cells and may or may not contain serum.

After the levels of the appropriate matrix proteins have stabilized, the ACG is incubated with an effective amount of an osteoarthritic phenotype inducing compound, with and without a test compound. The vehicles for delivery of the osteoarthritic phenotype and the test compound include any acceptable solvent in which the compositions or compounds are soluble, and that does not destroy the ACG tissue. After allowing an appropriate amount of time for the osteoarthritic phenotype to develop (e.g., 24-72 hours), the ACG is harvested. The method used to harvest the treated ACG will depend on the method selected for measuring the osteoarthritic markers, which in turn will depend on the markers chosen for evaluation. Cross sections of the ACG tissue are made when histological analysis will be used to measure markers. These sections can be harvested and prepared by a variety of known methods. For example, the cross-sections can be prepared by fixing total ACG tissue in a fixative agent and sectioning of the fixed tissue on a microtome or other sectioning device. When RNA analysis will be used, it is important to take the appropriate procedures to prevent degradation of the RNA, such as including RNAse inhibitors in the harvesting medium. When protein analysis will be performed, it is important to prevent degradation of the proteins by, for example, including protease inhibitors in the harvesting medium. If osteoarthritic markers are secreted into the medium, the medium can also be harvested.

After harvest of the tissue or medium, the levels of one or more osteoarthritic markers are determined. For each treatment group, one or more osteoarthritic markers can be measured in one or more ways. For example, aggrecanase RNA levels in an ACG treated with an osteoarthritic phenotype inducing composition and a test compound can be analyzed by quantitative RT-PCR. Analysis of collagen type II protein levels can be carried out by Western blot. Analysis of IL6 protein levels in the medium may be carried out by ELISA.

Levels of one or more osteoarthritic markers in ACGs incubated with the test compound are compared with levels of the same marker(s) in ACGs incubated with the osteoarthritic inducing composition alone. For the test compound to be considered efficacious, for example, RNA levels of aggrecanase 1 should be lower than those in ACGs with the osteoarthritic phenotype, and/or the protein levels of collagen type II should be higher than those in ACGs with the osteoarthritic phenotype, and/or the protein levels of IL6 should be lower than those in ACGs with the osteoarthritic phenotype. Alternatively, the change in the levels of the osteoarthritic markers in untreated ACGs and ACGs incubated with the osteoarthritic phenotype inducing composition alone can be compared to the change in the levels of the same markers in untreated ACGs and ACGs incubated with both the test compound and the osteoarthritic phenotype inducing composition. If the change in the level of the marker(s) (above the untreated ACG baseline) in ACG treated with both an osteoarthritic phenotype inducing composition and a test compound is less than the change in the level of the marker(s) in ACG treated with the osteoarthritic inducing composition alone, then the test compound is efficacious.

ACGs expressing the osteoarthritic phenotype can be used in the methods disclosed herein, or for other uses. For example, ACGs of the invention can be used for testing the effects of the cytokines on non-osteoarthritic marker molecules. It may also be used for testing the effects of the cytokines on other phenotypes and/or the effects of test compounds on those phenotypes. These could include cartilage growth or development, gene transcription,-post-translational processing of proteins, or protein degradation mechanisms. Other uses for the compositions of the invention are not limited to the examples disclosed herein.

The methods and assays of the invention will allow the identification of novel compositions with anti-osteoarthritic activity. Therefore, another aspect of the invention is a compound identified as anti-osteoarthritic by the claimed methods.

Other embodiments of the invention will be apparent to those skilled in the art from consideration of the specification and practice of the invention disclosed herein. It is intended that the specification and examples be considered as exemplary only.

### EXAMPLE 1

### Construction of Articular Cartilage Grafts (ACGs)

ACGs were constructed from the human chondrocyte strain Cc 05912 1°, which was isolated and propagated in Dubelcco's-Modified Eagle's Medium with. 10% fetal bovine serum (DMEM/10% FBS). After propagation, the cells were used to assemble ACGs on type II collagen coated Millipore Millicell-CM filters at a density of 2 million cells/cm², using the procedure described in U.S. Pat. No. 5,326,357. The ACGs were grown in defined ACG media (DMEM/2×ITS×(Invitrogen # 51500-056), 2% Human Serum Albumin) supplemented with 5 ng/ml TGF-β2 and 100 μg/ml ascorbic acid. After three weeks in culture, the levels of aggrecan and collagen type II expression in the ACGs peaked and leveled off.

### EXAMPLE 2

### Treatment of ACGs with IL-1β and DBH

After three weeks, the IL-1β (R&D Systems # 201-LB) and DBH incubations began. All of the preincubations and subsequent incubations were performed in DMEM. First all of the ACGs were washed twice with DMEM. Sample 1 ACGs were harvested for histology and gene expression analysis at the onset of the experiment. Next, 2 hour preincubations were performed on four groups, in triplicate for both histology and gene expression analysis. Sample 2 ACGs received 250 μl of DMEM to the inside and outside of the ACG. Sample 3 ACGs received the same. Sample 4 ACGs received 5 μm DBH in the same volumes as the other groups to the inside and outside of the Millicell filter containing the ACGs. Sample 5 ACGs received DMSO in the same volume as the DBH group as a vehicle control. The ACGs were then incubated for 2 hours. After the 2 hour preincubation with DBH, IL-1β incubation was added. Sample 2 received another 250 μl of DMEM in addition to the 250 μl that was added at the preincubation step. Sample 3 received 250 μl, inside and out, of DMEM with IL-1β at a final concentration of 2 ng/ml. Sample 4 also received IL-1β with the addition of more DBH to compensate for the volume change of DMEM to keep the concentration of 5 μm. Sample 5 also received IL-1β with an adjustment of the DMSO levels to compensate for the volume change of the DMEM.

### EXAMPLE 3

### Histological Assessment of Proteoglycan and Collagen Levels

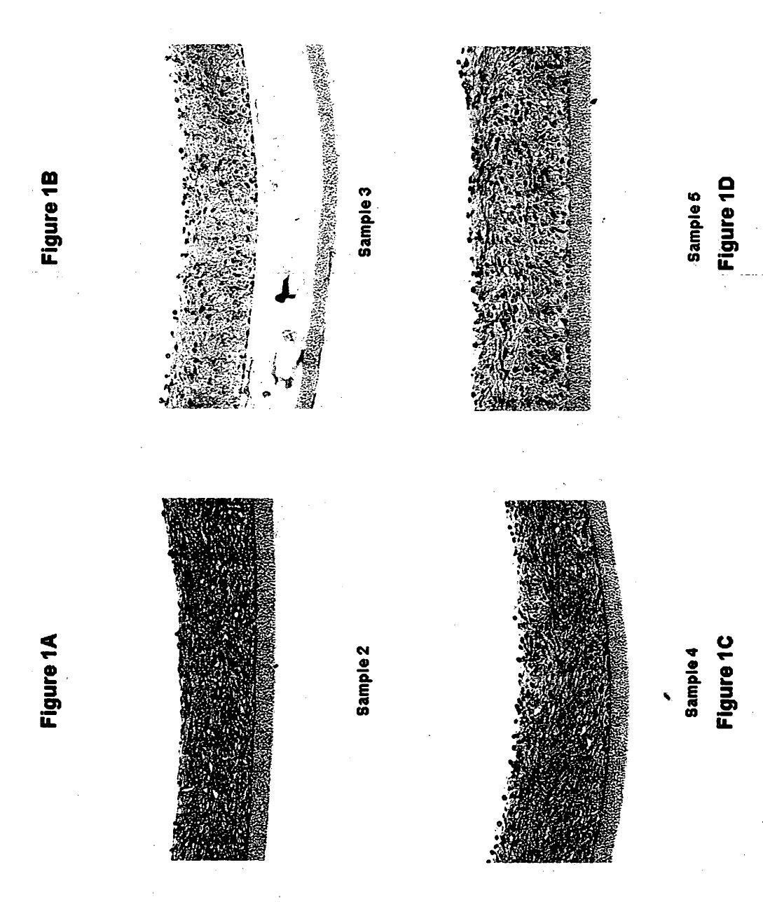

After incubation with-lL-1β for 24 hours, a group of the ACGs from Example 2 were fixed in 10% neutral buffered formalin and embedded in paraffin. Sections were cut and stained with toluidine blue to assess the presence or loss of proteoglycans in the ACGs. As shown in FIG. 1, ACGs treated with IL-1β show a marked loss of proteoglycans. Sections of the ACGs incubated with IL-1β and DBH showed decreased loss of proteoglycans.

### EXAMPLE 4

### Quantitative RT-PCR and Northern Blots

After incubation with IL-1β for 24 hours, a group of the ACGs from Example 2 were harvested for quantitative real-time RT-PCR by snap freezing them in LN₂. For quantitative real-time RT-PCR total RNA was isolated from the ACGs with TRIzol™ Reagent (Invitrogen # 15596-026) with subsequent cleanup using the Qiagen Rneasy™ Mini Kit (Qiagen # 74104). Contaminating DNA was removed from total RNA samples using the Ambion DNA-free kit (Ambion # 1906). cDNA synthesis was performed on 1 ™g of each sample using the Amersham Ready-To-Go You-Prime First-Strand Beads™ (Amersham # 27-9264-01) primed with oligo dT. Reverse transcriptase activity was measured using the PicoGreen™ dsDNA quantitation Kit (Molecular Probes # P-7589). Quantitative real-time PCR (Q RT-PCR) was performed using the ABI Prism™ Sequence Detection System (Applied Biosystems). PCR was performed on 2.5 μl of each RT reaction, after they were brought up to 100 μl with nuclease free H₂O, using TaqMan™ Universal PCR Master Mix (Applied Biosystems # 4304437).

The primers and probes set forth below were used in the Q RT-PCR at concentrations from 100 to 900 nm:

| | | |
|---|---|---|
| aggrecanase 1 | | |
| primers: | forward, 5′ actggtggtggcagatgaca 3′ | (SEQ ID NO: 1) |
| | reverse, 5′ tcactgttagcaggtagcgcttt 3′ | (SEQ ID NO: 2) |
| probe: | 5′ tggccgcattccacggtgc 3′ | (SEQ ID NO: 3) |
| aggrecanase 2 | | |
| primers: | forward, 5′ tggctcacgaaatcggaca 3′ | (SEQ ID NO: 4) |
| | reverse, 5′ ggaaccaaaggtctcttcacaga 3′ | (SEQ ID NO: 5) |
| probe: | 5′ cttggcctctcccatgacgattcca 3′ | (SEQ ID NO: 6) |
| collagenase 1 | | |
| primers: | forward, 5′ gggagatcatcgggacaactc 3′ | (SEQ ID NO: 7) |
| | reverse, 5′ gggcctggttgaaaagcat 3′ | (SEQ ID NO: 8) |
| probe: | 5′ ccttttgatggacctggaggaaatcttgc 3′ | (SEQ ID NO: 9) |
| collagenase 3 | | |
| primers: | forward, 5′ tggcattgctgacatcatga 3′ | (SEQ ID NO: 10) |
| | reverse, 5′ gccagagggcccatcaa 3′ | (SEQ ID NO: 11) |
| probe: | 5′ ctcttttggaattaaggagcatggcgactt 3′ | (SEQ ID NO: 12) |
| stromelysin | | |
| primers: | forward, 5′ ttctcgttgctgctcatgaaa 3′ | (SEQ ID NO: 13) |
| | reverse, 5′ tagagtgggtacatcaaagcttcagt 3′ | (SEQ ID NO: 14) |
| probe: | 5′ tggccactccctgggtctctttcac 3′ | (SEQ ID NO: 15) |

Thermal cycling conditions were as follows: 50° C for 2 minutes with amperase uracil-N-glycosylase (UNG) incubation to prevent reamplification of carryover PCR products. The next step was AmpliTaq Gold™ activation at 95° C. for 10 minutes. This was followed by PCR for 40 cycles consisting of 95° C. for 15 seconds to denature and 60° C. for 1 minute to anneal/extend. Gene expression was determined by absolute quantitation using plasmids of the previously mentioned genes for standard curves. Results from the quantitative real-time PCR were normalized to the results from the PicoGreen™ assay.

The results of the real time quantitative PCR studies are summarized below and presented in FIGS. 2-6.

| | | % reduction of IL-1β- |
|---|---|---|
| Osteoarthritic | Fold Induction | induced gene |
| marker | by IL-1β | expression with DBH |
| collagenase 1 | 183 | 66 |
| collagenase 3 | 17 | 55 |
| stromelysin | 168 | 57 |
| aggrecanase 1 | 4 | 65 |
| aggrecanase 2 | 4 | 56 |

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. An articular cartilage graft (ACG) having osteoarthritic cartilage damage.

2. An articular cartilage graft (ACG) comprising an ACG treated with an amount of one or more cytokines effective to induce osteoarthritic cartilage damage in the ACG.

3. The ACG of claim 2 wherein the one or more cytokines are selected from the group consisting of IL-1α, IL-1β, and TNFα.

4. A method for making the articular cartilage graft of claim 1 comprising treating the ACG with an amount of one or more cytokines effective to induce the osteoarthritic cartilage damage.

5. An assay for evaluating the efficacy of a test compound for treating or preventing osteoarthritic cartilage damage comprising:
(a) treating an ACG with an osteoarthritic cartilage damage inducing compound;
(b) treating the ACG with a test compound; and
(c) comparing the change in the levels of one or more osteoarthritic markers in an ACG before and after administration of an osteoarthritic phenotype inducing composition alone with the change in the levels of the same markers in an ACG before and after administration of both the test compound and the osteoarthritic phenotype inducing composition; wherein a lower level of change in the ACG to which the test compound was administered indicates that the test compound is efficacious for treating or preventing osteoarthritic cartilage damage.

6. The assay of claim 5 wherein the osteoarthritic cartilage damage inducing compound comprises one or more cytokines.

7. The assay of claim 6 wherein the one or more cytokines are selected from the group consisting of IL-1α, IL-1β, and TNFα.

8. The assay of claim 5 wherein the test compound is administered before the ostearthritic phenotype inducing composition;

9. The assay of claim 5 wherein the test compound is administered after the osteoarthritic phenotype inducing composition.

10. The assay of claim 5 wherein the test compound and the osteoarthritic phenotype inducing composition are administered at the same time.

11. The assay of claim 5 wherein at least one osteoarthritic marker is a matrix protein.

12. The assay of claim 5 wherein the matrix protein is a collagen or a proteoglycan.

13. The assay of claim 11 wherein the matrix protein is type II collagen.

14. The assay of claim 11 wherein the matrix protein is aggrecan.

15. The assay of claim 5 wherein at least one osteoarthritic marker is a metalloproteinase.

16. The assay of claim 15 wherein the metalloproteinase protein is selected from the group consisting of collagenase 1, collagenase 3, aggrecanase 1, aggrecanase 2, and stromelysin.

17. The assay of claim 5 wherein at least one osteoarthritic marker is an inflammatory protein.

18. The assay of claim 17 wherein the inflammatory protein is selected from the group consisting of TNF, IL-6, IL-8, IL-1β, nitric oxide synthase 2A, prostaglandin-endoperoxidase synthase 2, or NF-κB.

19. The assay of claim 5 wherein the levels of the osteoarthritic markers are determined by RNA levels.

20. The assay of claim 19 wherein the RNA levels are determined by RT-PCR or Northern Blot.

21. The assay of claim 5 wherein the levels of the osteoarthritic markers are determined by protein levels.

22. The assay of claim 21 wherein the protein levels are determined by an assay selected from the group consisting of histology, Western blot, ELISA, and immunohistochemistry.

23. The assay of claim 5 wherein the protein levels of a matrix protein and the RNA levels of a metalloproteinase are determined in the same ACG.

24. The assay of claim 5 wherein the protein levels of a matrix protein and the RNA levels of an inflammatory molecule are determined in the same ACG.
